(19) United States
(12) Patent Application Publication
(10) Pub. No.: US 2010/0319244 A1
(43) Pub. Date: Dec. 23, 2010

(54) SPECIFIC POLYISOBUTENEAMINES AND THEIR USE AS DETERGENTS IN FUELS

(75) Inventors: Erich K. Fehr, Vellmar (DE); Dietmar Posselt, Heidelberg (DE); Peter Spang, St. Ingbert (DE); Harald Schwahn, Heidelberg (DE); Marc Walter, Frankenthal (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/865,613

(22) PCT Filed: Jan. 29, 2009
(86) PCT No.: PCT/EP09/51010

§ 371 Date: Jul. 30, 2010

(30) Foreign Application Priority Data
Feb. 1, 2008 (EP) 08101217.1

(51) Int. Cl. C07C 211/20; C07C 209/00; C10L 1/223

(52) U.S. Cl. 44/412; 564/509; 564/467

(57) ABSTRACT

Polyisobuteneamines of the general formula R¹—CH₂—NR²R³ in which R¹ is a polyisobutyl radical which is derived from isobutene and up to 20% by weight of n-butene and has a number-average molecular weight Mₙ of from 600 to 770, and R² and R³ are each independently hydrogen, a C₁-C₁₈-alkyl, C₂-C₁₈-alkenyl, C₄-C₁₈-cycloalkyl, C₁-C₁₈-alkylaryl, hydroxy-C₁-C₁₈-alkyl, poly(oxyalkyl), polyalkylenepolyamine or polyalkyleneimine radical or, together with the nitrogen atom to which they are bonded, are a heterocyclic ring are suitable as detergents in gasoline fuels, reduce valve sticking and improve the compatibility of the detergents with carrier oils and compatibility in fuel compositions which comprise a mineral fuel content and C₁-C₄-alkanols.

The present invention relates to novel polyisobuteneamines of the general formula I

R¹—CH₂—NR²R³ (I)

in which
the variable R¹ is a polyisobutyl radical which is derived from isobutene and up to 20% by weight of n-butene and has a number-average molecular weight Mₙ of from 600 to 770, and
the variables R² and R³ are each independently hydrogen, a C₁-C₁₈-alkyl, C₂-C₁₈-alkenyl, C₄-C₁₈-cycloalkyl, hydroxy-C₁-C₁₈-alkyl, poly(oxyalkyl), polyalkylenepolyamine or polyalkyleneimine radical or, together with the nitrogen atom to which they are bonded, are a heterocyclic ring.

The present invention further relates to fuel compositions, especially those having a content of C₁-C₄-alkanols, which comprise the polyisobuteneamines in an amount effective as a detergent.

The present invention further relates to the use of these polyisobuteneamines as fuel additives for reducing valve sticking and/or for improving the compatibility of the detergents with carrier oils, especially at low temperatures, and/or for improving compatibility in fuel compositions which comprise a mineral fuel content and C₁-C₄-alkanols.

EP 0 244 616 A2 {1} discloses polybutyl- and polyisobuteneamines of the general formula R¹—CH₂—NR²R³ in which R is a polybutyl or polyisobutyl radical derived from isobutene and up to 20% by weight of n-butene and has a number-average molecular weight Mₙ of 300-5000, preferably of 500-2500 and, according to the experimental examples, of 900-1000. These polybutyl- and polyisobuteneamines can be obtained by hydroformylating the underlying poly(iso)butenes and subsequent hydrogenating amination of the oxo products present. They are recommended as fuel detergents with valve-cleaning or valve keep-clean action.

WO 2004/087808 A1 {2} describes formulations composed of polyalkeneamines and solvents with improved low-temperature properties, which are manifested in a lower cloud point, a lower pour point and/or an improved low-temperature storage stability of the formulation. The polyalkenes underlying these polyalkeneamines have a number-average molecular weight Mₙ of especially “from about 500 to about 5000 or from about 800 to 1200, or from 850 to 1100, for example about 1000”. This polyalkene is preferably a polyisobutene. A preferred process for preparing polyalkeneamines based on polyisobutene is the hydroformylation of the underlying polyisobutene and the subsequent reductive amination of the oxo intermediate. The specific b polyisobuteneamines disclosed in the experimental examples have number-average molecular weight Mₙ of 950 or 1000. Such formulations composed of polyalkeneamines and solvents can be used as additives in gasoline fuels, especially for improving the intake system-cleaning action of gasoline fuels, in which case these gasoline fuels may also comprise predominant amounts of C₁-C₄-alkanols, for example 15% by volume of methanol, 65% by volume of ethanol, 20% by volume of isopropanol, 15% by volume of tert-butanol or 20% by volume of isobutanol.

US 2006/0277820 A1 {3} discloses additives for controlling deposits in gasoline engines, which comprise a mixture of polyisobuteneamines of mean molecular weight from about 700 to 1000, especially of about 800 (though it is unclear whether this is the number-average or the weight-average molecular weight), and Mannich bases. No details of the structure or preparation method of the polyisobuteneamines are given; the indication of the source of the polyisobuteneamine “PURAD 6847/2 [BASF, Germany]” is not based on a commercial product available to the public.

However, the polyisobuteneamine fuel detergents known from the prior art are still in need of improvement in terms of their spectrum of action. Although they generally have satisfactory action in the cleaning and keeping-clean of the intake valves and of the intake system of the engines, they still have deficits in the reduction of valve sticking, in their action with regard to the compatibility of the detergents with carrier oils, especially at low temperatures, and/or in their action with regard to compatibility in fuel compositions which comprise a mineral fuel content and C₁-C₄-alkanols. Moreover, the known polyisobuteneamines are usually too viscous, such that capacity bottlenecks exist in their preparation owing to the limited flow rates through the apparatus and lines.

It was therefore an object of the present invention, in a first aspect, to provide novel polyisobuteneamines as fuel additives which, as well as a satisfactory action in the cleaning and keeping-clean of the intake valves and of the intake system of the engines, reduce valve sticking.

It was therefore an object of the present invention, in a second aspect, to provide novel polyisobuteneamines as fuel additives which, as well as a satisfactory action in the cleaning and keeping-clean of the intake valves and of the intake system of the engines, bring about an improvement in the compatibility of the detergents with carrier oils, in particular with polyether and polyetheramine carrier oils, especially at low temperatures.

It was therefore an object of the present invention, in a third aspect, to provide novel polyisobuteneamines as fuel additives which, as well as a satisfactory action in the cleaning and keeping-clean of the intake valves and of the intake system of the engines, bring about an improvement in compatibility in fuel compositions which comprise a mineral fuel content and C₁-C₄-alkanols. “Mineral fuel content” shall be understood here to mean the hydrocarbon-based fuel components which stem from the underlying mineral oil or the synthetically obtained fuel components.

It was therefore an object of the present invention, in a fourth aspect, to provide novel polyisobuteneamines as fuel additives which, as well as a satisfactory action in the cleaning and keeping-clean of the intake valves and of the intake system of the engines, simultaneously to reduce valve sticking, to improve the compatibility of the detergents with carrier oils, in particular with polyether and polyetheramine carrier oils, especially at low temperatures, and to improve compatibility in fuel compositions which comprise a mineral fuel content and C₁-C₄-alkanols.

It was therefore an object of the present invention, in a fifth aspect, to provide novel polyisobuteneamines as fuel additives which, as well as a satisfactory action in the cleaning and keeping-clean of the intake valves and of the intake system of the engines, simultaneously reduce valve sticking, improve the compatibility of the detergents with carrier oils, in particular with polyether and polyetheramine carrier oils, especially at low temperatures, improve compatibility in fuel compositions which comprise a mineral fuel content and C₁-C₄-alkanols, and are at the same time sufficiently mobile (i.e. have a sufficiently low viscosity) that capacity bottlenecks in their preparation owing to limited flow rates through the apparatus and lines are avoided.

Accordingly, the novel polyisobuteneamines of the general formula I defined at the outset and their use as fuel additives for remedying the deficiencies detailed above in the spectrum of action of polyisobuteneamine fuel detergents have been found.

The polyisobutyl radical R¹ in the general formula I derives from isobutene and up to 20% by weight, preferably up to 10% by weight, especially up to 5% by weight, in particular up to 2% by weight, of n-butene. n-Butene shall be understood here to mean all linear, ethylenically unsaturated C₄-hydrocarbons, especially 2-butene and in particular 1-butene. The polyisobutyl radical R¹ can also be derived from isobutene alone. The R¹ radical is thus a more or less regularly branched polymer chain which consists predominantly of repeat units of the formula —CH₂—C(CH₃)₂—CH₂—C(CH₃)₂—, and units with longer linear moieties of the formula —CH₂—(CH₃)₂—(CH₂)₄— can also occur in the case of incorporation of 1-butene.

The variable R¹ has a number-average molecular weight Mₙ of from 600 to 770, especially from 650 to 750, in particular from 700 to 730. A typical value here is Mₙ=720. The number-average molecular weight Mₙ is known to be defined as the ratio of the mass of a polymer to the number of molecules present therein, i.e. the measurement depends on the number of macromolecules and not on their size. The number-average molecular weight Mₙ is typically determined by vapor pressure osmometry or cryometry. In contrast, the weight-average molecular weight M_{w} depends on the size of the macromolecules. The weight-average molecular weight M_{w} is typically determined by light scattering or the sedimentation equilibrium. With regard to the mathematical definitions of Mₙ and M_{w} and the performance of the experimental determination methods for Mₙ and M_{w}, reference is made to the relevant technical knowledge.

In a preferred embodiment, the polyisobutyl radical for the variable R¹ has been obtained from a polyisobutene which has at least one of the following properties:

[a] proportion of vinylidene double bonds of at least 60 mol %, preferably of at least 70 mol %, especially of at least 80 mol %, in particular of at least 85 mol %, based in each case on the polyisobutene;
[b] content of isobutene units in the polyisobutene polymer skeleton of at least 85% by weight, preferably of at least 90% by weight, especially of at least 95% by weight, in particular of at least 98% by weight;
[c] polydispersity of from 1.05 to 7, preferably from 1.1 to 2.5, especially from 1.1 to less than 1.9, in particular from 1.1 to less than 1.5.

The polyisobutene used to obtain the polyisobutyl radical for the variable R¹ preferably simultaneously has properties [a] and [b] or simultaneously has properties [a] and [c] or simultaneously has properties [b] and [c] or simultaneously has properties [a], [b] and [c].

The abovementioned polyisobutenes with properties [a] and/or [b] and/or [c] are generally so-called “high-reactivity” polyisobutenes which are notable especially for a high content of terminal double bonds, i.e. alpha-olefinic vinylidene double bonds. Suitable high-reactivity polyisobutenes are, for example, polyisobutenes which have a proportion of vinylidene double bonds of at least 60 mol %, preferably of at least 70 mol %, especially of at least 80 mol %, in particular of at least 85 mol %. Preference is also given to polyisobutenes which have predominantly homogeneous polymer skeletons. Predominantly homogeneous polymer skeletons are possessed especially by those polyisobutenes which are formed from isobutene units to an extent of at least 85% by weight, preferably to an extent of at least 90% by weight, especially to an extent of at least 95% by weight, in particular to an extent of at least 98 mol %. In addition, the high-reactivity polyisobutenes normally have a polydispersity in the range from 1.05 to 7, preferably from 1.1 to 2.5, especially from 1.1 to less than 1.9, in particular from 1.1 to less than 1.5. Polydispersity is understood to mean the quotient of weight-average molecular weight M_{w} divided by the number-average molecular weight Mₙ.

To prepare the inventive polyisobuteneamines of the general formula I, the high-reactivity polyisobutenes mentioned are preferably reacted with carbon monoxide and hydrogen in a hydroformylation reaction in the presence of a hydroformylation catalyst, for example of a rhodium or cobalt catalyst, and if appropriate of suitable inert solvents, for example hydrocarbons, at typically from 80 to 200° C. and CO/H₂ pressures of up to 600 bar, and the oxo intermediates thus prepared are subjected to a reductive amination in the presence of hydrogen, of a suitable nitrogen compound, of a suitable catalyst, for example Raney nickel or Raney cobalt, and if appropriate of suitable inert solvents, for example alcohols and/or hydrocarbons, at typically from 80 to 200° C. and hydrogen pressures of up to 600 bar, especially from 80 to 300 bar. The —CH₂— moiety in the formula I which occurs as a bridging member between polyisobutyl radical R¹ and nitrogen-containing moiety —NR²R³ and is partly responsible for the structural properties results from the carbon monoxide supplied in the hydroformylation stage.

The hydroformylation and reductive amination steps mentioned for obtaining the inventive polyisobuteneamines I are very well known to those skilled in the art and are described in detail, for example, in {1}. The preparation of the high-reactivity polyisobutenes used for this purpose is likewise very well known to those skilled in the art; it is preferably done by cationic polymerization of pure isobutene or of a technical C₄ hydrocarbon stream which is rich in isobutene and additionally comprises essentially 1-butene, 2-butene and butanes, for example raffinate I, in the presence of boron trifluoride or of a boron trifluoride complex as a catalyst.

Suitable amines, from which the nitrogen-containing moiety —NR²R³ in the general formula I derives and which can be used in the above-described hydroformylation reaction to prepare the inventive polyisobuteneamines are compounds of the formula HNR²R³. The variables R² and R³ therein are the same or are independent of one another and are each:

(1) hydrogen;
(2) a C₁-C₁₈-alkyl radical; examples of suitable alkyl radicals include straight-chain or branched alkyl radicals having from 1 to 18 carbon atoms, such as methyl, ethyl, iso- or n-propyl, n-, iso-, sec- or tert-butyl, n- or isopentyl; and also n-hexyl, n-heptyl, n-octyl, n-nonyl, n-decyl, n-undecyl, n-tridecyl, n-tetradecyl, n-pentadecyl, n-hexadecyl and n-octadecyl and the singularly or multiply branched analogs thereof; and corresponding radicals in which the carbon chain has one or more ether bridges;
(3) a C₂-C₁₈-alkenyl radical; examples of suitable alkenyl radicals include the mono- or polyunsaturated, preferably mono- or diunsaturated, analogs of the above-mentioned alkyl radicals having from 2 to 18 carbon atoms, where the double bond may be in any position in the carbon chain;
(4) a C₄-C₁₈-cycloalkyl radical; examples include cyclobutyl, cyclopentyl and cyclohexyl, and the analogs thereof substituted by from 1 to 3 C₁-C₄-alkyl radicals, where the C₁-C₄-alkyl radicals are preferably selected from methyl, ethyl, iso- or n-propyl, n-, iso-, sec- or tert-butyl;
(5) a (C₁-C₁₈-alkyl)aryl radical where the C₁-C₁₈-alkyl group is as defined above and the aryl group is derived from mono- or bicyclic, fused or nonfused, 4-7-membered, especially 6-membered, aromatic or heteroaromatic groups such as phenyl, pyridyl, naphthyl and biphenylyl;
(6) a (C₂-C₁₈-alkenyl)aryl radical where the C₂-C₁₈-alkenyl group is as defined above and the aryl group is likewise as defined above;
(7) a hydroxy-C₁-C₁₈-alkyl radical which corresponds to the mono- or polyhydroxylated, preferably monohydroxylated, especially terminally monohydroxylated, analogs of the above C₁-C₁₈-alkyl radicals, for example 2-hydroxyethyl and 3-hydroxypropyl;
(8) an optionally hydroxylated poly(oxyalkyl) radical which is obtainable by alkoxylating the nitrogen atom having from 2 to 10 C₁-C₄-alkoxy groups, where individual carbon atoms may optionally bear further hydroxyl groups; preferred alkoxy groups comprise methoxy, ethoxy and n-propoxy groups;
(9) a polyalkylenepolyamine radical of the formula

Z—NH—(C₁-C₆-alkylene-NH)ₘ—C₁-C₆-alkylene-

in which m is an integer from 0 to 5, Z is hydrogen or C₁-C₆-alkyl, and C₁-C₆-alkyl denotes radicals such as methyl, ethyl, iso- or n-propyl, n-, iso-, sec- or tert-butyl, n- or isopentyl or n-hexyl, and C₁-C₆-alkylene represents the corresponding bridging analogs of these radicals;
(10) a polyalkyleneimine radical formed from 1 to 10 C₁-C₄-alkyleneimine groups, especially ethyleneimine groups; or
(11) together with the nitrogen atom to which they are bonded, are optionally substituted 5 to 7-membered heterocyclic ring which is optionally substituted by from one to three C₁-C₄-alkyl radicals and optionally bears a further ring heteroatom such as O or N.

Typical examples of suitable compounds of the formula HNR²R³ are:
ammonia;
primary amines such as methylamine, ethylamine, n-propylamine, isopropylamine, n-butylamine, isobutylamine, sec-butylamine, tert-butylamine, pentylamine, hexylamine, cyclopentylamine and cyclohexylamine; and primary amines with ether oxygen or hydroxyl functions of the formula CH₃—O—C₂H₄—NH₂, C₂H₅—O—C₂H₄—NH₂, CH₃—O—C₃H₆—NH₂, C₂H₆—O—C₃H₆—NH₂, n-C₄H₉—O—C₄H₈—NH₂, HO—C₂H₄—NH₂, HO—C₃H₆—NH₂ and HO—C₄H₈—NH₂;
secondary amines, for example dimethylamine, diethylamine, methylethylamine, di-n-propylamine, diisopropylamine, diisobutylamine, di-sec-butylamine, di-tert-butylamine, dipentylamine, dihexylamine, dicyclopentylamine, dicyclohexylamine and diphenylamine; and secondary amines with ether oxygen or hydroxyl functions of the formula (CH₃—O—C₂H₄)₂NH, (C₂H₅—O—C₂H₄)₂NH, (CH₃—O—C₃H₆)₂NH, (C₂H₆—O—C₃H₆)₂NH, (n-C₄H₉—O—C₄H₈)₂NH, (HO—C₂H₄)₂NH, (HO—C₃H₆)₂NH and (HO—C₄H₈)₂NH;
heterocyclic amines such as pyrrolidine, piperidine, morpholine and piperazine, and substituted derivatives thereof, such as N—C₁-C₆-alkylpiperazines and dimethylmorpholine;
polyamines, for example C₁-C₄-alkylenediamines, di-C₁-C₄-alkylenetriamines, tri-C₁-C₄-alkylenetetramines and higher analogs; and polyethyleneimines, preferably oligoethyleneimines, consisting of from 1 to 10 and preferably from 2 to 6 ethyleneimine units; examples of suitable polyamines and polyimines are n-propylenediamine, 1,4-butanediamine, 1,6-hexanediamine, diethylenetriamine, triethylenetetramine, tetraethylenepentamine, pentaethylenehexamine, hexaethyleneheptamine and polyethyleneimines, and also alkylation products thereof, for example 3-(dimethylamino)-n-propylamine, N,N-dimethylethylenediamine, N,N-diethylethylenediamine and N,N,N′,N′-tetramethyldiethylenetriamine; likewise suitable is ethylenediamine.

In a particularly preferred embodiment, the present invention relates to polyisobuteneamines of the general formula I in which the —NR²R³ moiety has been obtained from ammonia or a polyamine of the general formula II

H₂N—(CH₂CH₂—NH—)ₙ—H (II)

in which the variable n is an integer from 1 to 5.

In a further particularly preferred embodiment, the present invention relates to polyisobuteneamines of the general formula I with a kinematic viscosity of from 70 to 200 cSt, especially from 80 to 150 cSt, in particular from 90 to 120 cSt, in each case measured in undiluted form at 100° C. Such viscosity values for the polyisobuteneamines I are often in the range from 95 to 105 cSt. The kinematic viscosities are typically measured here in an Ubbelohde viscosimeter.

The totality of all structural features of these polymers is important for the establishment of the comparatively low viscosity of the inventive polyisobuteneamines of the general formula I. Influencing parameters are the length (expressed by the number-average molecular weight Mₙ), the regularity of the branches of the polymer chain and their attachment site to the —CH₂—NR²R³ moiety. It thus makes a difference whether the polymer chain is formed only from isobutene units (i.e. has a regular branching pattern) or whether linear n-butene units (as a disruption to the branching pattern) are also incorporated. In addition, the polydispersity (i.e. the quotient of weight-average molecular weight and number-average molecular weight M_{w}/Mₙ) also exerts an influence on the viscosity of the polymer. A further influence results from the type and size of the NR²R³ moiety on the polymer chain. To establish the desired viscosity range, an adjustment of all structural features mentioned with respect to one another is necessary in the context of the above definitions of these structural features. This adjustment is not forecastable or precalculable.

In addition to the pure mechanical advantage of better flow through apparatus and lines, the viscosity also exerts an influence, in an unforeseeable, favorable manner, on the mode of action of the inventive polyisobuteneamines of the general formula I as fuel additives. For instance, the polyisobuteneamines I have a further enhanced action in the reduction of valve sticking, in the improvement of the compatibility of the detergents with carrier oils, especially at low temperatures, and in the improvement of compatibility in fuel compositions which comprise a mineral fuel content and C₁-C₄-alkanols when they have a kinematic viscosity of from 70 to 200 cSt, especially from 80 to 150 cSt, in particular from 90 to 120 cSt, in each case measured in undiluted form at 100° C., without this impairing their good action in the cleaning and keeping-clean of the intake valves and of the intake system of the engines.

The inventive polyisobuteneamines of the general formula I are outstandingly suitable as fuel additives with detergent action. Therefore, the present invention also provides fuel compositions, especially those having a content of C₁-C₄-alkanols, which comprise at least one polyisobuteneamine of the general formula I in an amount effective as a detergent. In addition to their satisfactory to outstanding action in the cleaning and keeping-clean of the intake valves and of the intake system of the engines, they additionally exert a series of further advantageous effects as fuel additives: they reduce valve sticking and/or they improve the compatibility of the detergents with carrier oils, in particular polyether and polyetheramine carrier oils, especially at low temperatures, and/or they improve compatibility in fuel compositions which comprise a mineral fuel content and C₁-C₄-alkanols. They are not least sufficiently mobile (i.e. they have a sufficiently low viscosity) that capacity bottlenecks in their preparation owing to limited flow rates through the apparatus and lines—even in the case of additional use of inert solvents or diluents—are avoided; the comparatively low viscosity also has an effect, in an unforeseeable, favorable manner, on their mode of action as fuel additives.

The present invention therefore also provides for the use of the inventive polyisobuteneamines of the general formula I as fuel additives for reducing valve sticking. “Valve sticking” is understood by those skilled in the art to mean that the valves, owing to the adherence of tacky residues, especially of fuel detergents, no longer close onto the valve shafts, such that the engine can only be started with a delay, if at all.

The present invention therefore further provides for the use of the inventive polyisobuteneamines of the general formula I as fuel additives for improving the compatibility of the detergents with carrier oils, in particular polyether and polyetheramine carrier oils, especially at low temperatures. When there is insufficient compatibility of detergents with carrier oils in the sense of storage stability of homogeneously prepared mixtures thereof, phase separations occur at low temperatures or cloudiness occurs even at room temperature. Low temperatures shall be understood here to mean the temperatures to which fuel additive packages and fuel additivized with them are exposed in the course of storage and transport; this is typically the temperature range from +10° C. to −25° C., especially from 0° C. to −20° C. In the case of storage-unstable mixtures, it is of course possible to add solvents, for example hydrocarbons such as xylene, as solubilizers—for economic reasons, such solvent additions should of course be avoided.

The present invention therefore further also provides for the use of the inventive polyisobuteneamines of the general formula I as fuel additives for improving compatibility in fuel compositions which comprise a mineral fuel content and C₁-C₄-alkanols. When there is insufficient compatibility of the fuel additives with the mineral fuel content and the lower alcohols mentioned in the sense of stability of homogeneously prepared mixtures thereof, cloudiness occurs or homogeneous mixtures cannot be prepared at all. This technical problem occurs especially in the case of use of fuels composed of a mineral content and very predominant amounts of lower alcohol, which will become ever more important in the future; one example of such a fuel is “E85”, a mixture of 85% by volume of ethanol and 15% by volume of mineral gasoline fuel.

The present invention therefore further also provides for the use of the inventive polyisobuteneamines of the general formula I as fuel additives for simultaneously reducing valve sticking, improving the compatibility of the detergents with carrier oils, in particular polyether and polyetheramine carrier oils, especially at low temperatures, and improving compatibility in fuel compositions which comprise a mineral fuel content and C₁-C₄-alkanols.

In connection with the present invention, fuel compositions are preferably understood to mean gasoline fuels. Useful gasoline fuels include all commercial gasoline fuel compositions. As a typical representative, mention shall be made here of the Eurosuper base fuel to EN 228, which is customary on the market. Further possible fields of use for the inventive polyisobuteneamines I are also gasoline fuel compositions of the specification according to WO 00/47698 {4}.

One example is a gasoline fuel composition with an aromatics content of not more than 60% by volume, for example not more than 42% by volume, and a sulfur content of not more than 2000 ppm by weight, for example not more than 150 ppm by weight.

The aromatics content of the gasoline fuel composition is preferably not more than 50% by volume, especially from 1 to 45% by volume, in particular from 5 to 40% by volume. The sulfur content of the gasoline fuel is preferably not more than 500 ppm by weight, especially from 0.5 to 150 ppm by weight, in particular from 1 to 100 ppm by weight.

In addition, the gasoline fuel composition may, for example, have an olefin content of up to 50% by volume, preferably from 0.1 to 21% by volume, especially from 2 to 18% by volume, a benzene content of up to 5% by volume, preferably from 0 to 1.0% by volume, especially from 0.05 to 0.9% by volume, and/or an oxygen content of up to 47.5% by weight, for example from 0.1 to 2.7% by weight, or, for example, from 2.7 to 47.5% by weight (for gasoline fuel compositions which comprise predominantly lower alcohols).

In particular, gasoline fuel compositions mentioned by way of example may also be those which simultaneously have an aromatics content of not more than 38% by volume, an olefin content of not more than 21% by volume, a sulfur content of not more than 50 ppm by weight, a benzene content of not more than 1.0% by volume and an oxygen content of from 0.1 to 47.5% by weight.

The summer vapor pressure of the gasoline fuel composition is typically not more than 70 kPa, especially 60 kPa (in each case at 37° C.).

The RON of the gasoline fuel composition is generally from 75 to 105. A customary range for the corresponding MON is from 65 to 95.

The specifications mentioned are determined by customary methods (DIN EN 228).

In addition to the use in gasoline fuels, however, use of the inventive polyisobuteneamines I in other fuel types, for example diesel fuels, kerosene or turbine fuels, is also possible in principle. Use in lubricant compositions is also conceivable.

In a preferred embodiment, the inventive fuel compositions, especially gasoline fuel compositions, comprise from 0.1 to 95% by volume, more preferably from 1 to 90% by volume, even more preferably from 5 to 90% by volume, especially from 10 to 90% by volume, in particular from 50 to 90% by volume, of C₁-C₄-alkanols as lower alcohol fuel components. Such fuels are described, for example, in WO 2004/090079 {5}. Useful C₁-C₄-alkanols include methanol, n-propanol, isopropanol, n-butanol, isobutanol, sec-butanol, tert-butanol and especially ethanol; mixtures of the C₁-C₄-alkanols mentioned are also possible as lower alcohol fuel components. In addition to the lower alcohol fuel components mentioned, the inventive fuel composition may also comprise ethers having 5 or more carbon atoms, for example methyl-tert-butyl ether, in the molecule in an amount of up to 30% by volume.

The inventive polyisobuteneamines of the general formula I can be added to the fuel compositions to be additivized individually or in a mixture with further active additive components (coadditives).

Examples of such coadditives may be additives having detergent action and/or having valve seat wear-inhibiting action other than the inventive polyisobuteneamines I (referred to together hereinafter as detergent additives). Such a detergent additive has at least one hydrophobic hydrocarbon radical having a number-average molecular weight (Mₙ) of from 85 to 20 000 and at least one polar moiety which is selected from:

(a) mono- or polyamino groups having up to 6 nitrogen atoms, at least one nitrogen atom having basic properties;
(b) nitro groups, if appropriate in combination with hydroxyl groups;
(c) hydroxyl groups in combination with mono- or polyamino groups, at least one nitrogen atom having basic properties;
(d) carboxyl groups or their alkali metal or alkaline earth metal salts;
(e) sulfonic acid groups or their alkali metal or alkaline earth metal salts;
(f) polyoxy-C₂-C₄-alkylene moieties which are terminated by hydroxyl groups, mono- or polyamino groups, at least one nitrogen atom having basic properties, or by carbamate groups;
(g) carboxylic ester groups;
(h) moieties which derive from succinic anhydride and have hydroxyl and/or amino and/or amido and/or imido groups; and/or
(i) moieties obtained by Mannich reaction of substituted phenols with aldehydes and mono- or polyamines.

The hydrophobic hydrocarbon radical in the above detergent additives, which ensures the adequate solubility in the fuel, has a number-average molecular weight (Mₙ) of from 85 to 20 000, especially from 113 to 10 000, in particular from 300 to 5000. Typical hydrophobic hydrocarbon radicals, especially in conjunction with the polar moieties (a), (c), (h) and (i), include the polypropenyl, polybutenyl and polyisobutenyl radical, each having Mₙ=from 300 to 5000, especially from 500 to 2500, in particular from 700 to 2300.

Examples of the above groups of detergent additives include the following:

Additives comprising mono- or polyamino groups (a) are preferably polyalkenemono- or polyalkenepolyamines based on polypropene or conventional (i.e. having predominantly internal double bonds) polybutene or polyisobutene having Mₙ=from 300 to 5000. When polybutene or polyisobutene having predominantly internal double bonds (usually in the beta- and gamma-position) are used as starting materials in the preparation of the additives, a possible preparative route is by chlorination and subsequent amination or by oxidation of the double bond with air or ozone to give the carbonyl or carboxyl compound and subsequent amination under reductive (hydrogenating) conditions. The amines used here for the amination may be, for example, ammonia, monoamines or polyamines, such as dimethylaminopropylamine, ethylenediamine, diethylenetriamine, triethylenetetramine or tetraethylenepentamine. Corresponding additives based on polypropene are described in particular in WO-A-94/24231.

Further preferred additives comprising monoamino groups (a) are the hydrogenation products of the reaction products of polyisobutenes having an average degree of polymerization P of from 5 to 100 with nitrogen oxides or mixtures of nitrogen oxides and oxygen, as described in particular in WO-A-97/03946.

Further preferred additives comprising monoamino groups (a) are the compounds obtainable from polyisobutene epoxides by reaction with amines and subsequent dehydration and reduction of the amino alcohols, as described in particular in DE-A-196 20 262.

Additives comprising nitro groups (b), if appropriate in combination with hydroxyl groups, are preferably reaction products of polyisobutenes having an average degree of polymerization P=from 5 to 100 or from 10 to 100 with nitrogen oxides or mixtures of nitrogen oxides and oxygen, as described in particular in WO-A-96/03367 and WO-A-96/03479. These reaction products are generally mixtures of pure nitropolyisobutenes (e.g. alpha,beta-dinitropolyisobutene) and mixed hydroxynitropolyisobutenes (e.g. alpha-nitro-beta-hydroxypolyisobutene).

Additives comprising hydroxyl groups in combination with mono- or polyamino groups (c) are in particular reaction products of polyisobutene epoxides obtainable from polyisobutene having preferably predominantly terminal double bonds and Mₙ=from 300 to 5000, with ammonia or mono- or polyamines, as described in particular in EP-A-476 485.

Additives comprising carboxyl groups or their alkali metal or alkaline earth metal salts (d) are preferably copolymers of C₂-C₄₀-olefins with maleic anhydride which have a total molar mass of from 500 to 20 000 and some or all of whose carboxyl groups have been converted to the alkali metal or alkaline earth metal salts and any remainder of the carboxyl groups has been reacted with alcohols or amines. Such additives are disclosed in particular by EP-A-307 815. Such additives serve mainly to prevent valve seat wear and can, as described in WO-A-87/01126, advantageously be used in combination with customary fuel detergents such as poly(iso)buteneamines or polyetheramines.

Additives comprising sulfonic acid groups or their alkali metal or alkaline earth metal salts (e) are preferably alkali metal or alkaline earth metal salts of an alkyl sulfosuccinate, as described in particular in EP-A-639 632. Such additives serve mainly to prevent valve seat wear and can be used advantageously in combination with customary fuel detergents such as poly(iso)buteneamines or polyetheramines.

Additives comprising polyoxy-C₂-C₄-alkylene moieties (f) are preferably polyethers or polyether amines which are obtainable by reaction of C₂-C₆₀-alkanols, C₆-C₃₀-alkanediols, mono- or di-C₂-C₃₀-alkylamines, C₁-C₃₀-alkylcyclohexanols or C₁-C₃₀-alkylphenols with from 1 to 30 mol of ethylene oxide and/or propylene oxide and/or butylene oxide per hydroxyl group or amino group and, in the case of the polyether amines, by subsequent reductive amination with ammonia, monoamines or polyamines. Such products are described in particular in EP-A-310 875, EP-A-356 725, EP-A-700 985 and U.S. Pat. No. 4,877,416. In the case of polyethers, such products also have carrier oil properties. Typical examples of these are tridecanol butoxylates, isotridecanol butoxylates, isononylphenol butoxylates and polyisobutenol butoxylates and propoxylates and also the corresponding reaction products with ammonia.

Additives comprising carboxylic ester groups (g) are preferably esters of mono-, di- or tricarboxylic acids with long-chain alkanols or polyols, in particular those having a minimum viscosity of 2 mm²/s at 100° C., as described in particular in DE-A-38 38 918. The mono-, di- or tricarboxylic acids used may be aliphatic or aromatic acids, and particularly suitable ester alcohols or ester polyols are long-chain representatives having, for example, from 6 to 24 carbon atoms. Typical representatives of the esters are adipates, phthalates, isophthalates, terephthalates and trimellitates of isooctanol, of isononanol, of isodecanol and of isotridecanol. Such products also have carrier oil properties.

Additives comprising moieties derived from succinic anhydride and having hydroxyl and/or amino and/or amido and/or imido groups (h) are preferably corresponding derivatives of polyisobutenylsuccinic anhydride which are obtainable by reacting conventional or highly reactive polyisobutene having Mₙ=from 300 to 5000 with maleic anhydride by a thermal route or via the chlorinated polyisobutene. Particular interest attaches to derivatives with aliphatic polyamines such as ethylenediamine, diethylenetriamine, triethylenetetramine or tetraethylenepentamine. Such fuel additives are described in particular in U.S. Pat. No. 4,849,572.

Additives comprising moieties (i) obtained by Mannich reaction of substituted phenols with aldehydes and mono- or polyamines are preferably reaction products of polyisobutene-substituted phenols with formaldehyde and mono- or polyamines such as ethylenediamine, diethylenetriamine, triethylenetetramine, tetraethylenepentamine or dimethylaminopropylamine. The polyisobutenyl-substituted phenols may stem from conventional or highly reactive polyisobutene having Mₙ=from 300 to 5000. Such “polyisobutene-Mannich bases” are described in particular in EP-A-831 141.

For a more precise definition of the fuel additives detailed individually, reference is explicitly made here to the disclosures of the abovementioned prior art documents.

The inventive polyisobuteneamines I can additionally be combined with further customary components and additives. These primarily include carrier oils without marked detergent action.

Suitable mineral carrier oils are the fractions obtained in crude oil processing, such as kerosene or naphtha, brightstock or base oils having viscosities, for example, from the SN 500-2000 class; but also aromatic hydrocarbons, paraffinic hydrocarbons and alkoxyalkanols. Likewise useful is a fraction which is obtained in the refining of mineral oil and is known as “hydrocrack oil” (vacuum distillate cut having a boiling range of from about 360 to 500° C., obtainable from natural mineral oil which has been catalytically hydrogenated under high pressure and isomerized and also deparaffinized). Likewise suitable are mixtures of abovementioned mineral carrier oils.

Examples of synthetic carrier oils usable in accordance with the invention are selected from: polyolefins (poly-alpha-olefins or poly(internal olefin)s), (poly)esters, (poly)alkoxylates, polyethers, aliphatic polyether amines, alkylphenol-started polyethers, alkylphenol-started polyether amines and carboxylic esters of long-chain alkanols.

Examples of suitable polyolefins are olefin polymers having Mₙ=from 400 to 1800, in particular based on polybutene or polyisobutene (hydrogenated or unhydrogenated).

Examples of suitable polyethers or polyetheramines are preferably compounds comprising polyoxy-C₂-C₄-alkylene moieties which are obtainable by reacting C₂-C₆₀-alkanols, C₆-C₃₀-alkanediols, mono- or di-C₂-C₃₀-alkylamines, C₁-C₃₀-alkylcyclo-hexanols or C₁-C₃₀-alkylphenols with from 1 to 30 mol of ethylene oxide and/or propylene oxide and/or butylene oxide per hydroxyl group or amino group, and, in the case of the polyetheramines, by subsequent reductive amination with ammonia, monoamines or polyamines. Such products are described in particular in EP-A-310 875, EP-A-356 725, EP-A-700 985 and U.S. Pat. No. 4,877,416. For example, the polyetheramines used may be poly-C₂-C₆-alkylene oxide amines or functional derivatives thereof. Typical examples thereof are tridecanol butoxylates or isotridecanol butoxylates, isononylphenol butoxylates and also polyisobutenol butoxylates and propoxylates, and also the corresponding reaction products with ammonia.

Examples of carboxylic esters of long-chain alkanols are in particular esters of mono-, di- or tricarboxylic acids with long-chain alkanols or polyols, as described in particular in DE-A-38 38 918. The mono-, di- or tricarboxylic acids used may be aliphatic or aromatic acids; suitable ester alcohols or polyols are in particular long-chain representatives having, for example, from 6 to 24 carbon atoms. Typical representatives of the esters are adipates, phthalates, isophthalates, terephthalates and trimellitates of isooctanol, isononanol, isodecanol and isotridecanol, for example di(n- or isotridecyl) phthalate.

Further suitable carrier oil systems are described, for example, in DE-A-38 26 608, DE-A-41 42 241, DE-A-43 09 074, EP-A-0 452 328 and EP-A-0 548 617, which are explicitly incorporated herein by way of reference.

Examples of particularly suitable synthetic carrier oils are alcohol-started polyethers having from about 5 to 35, for example from about 5 to 30, C₃-C₆-alkylene oxide units, for example selected from propylene oxide, n-butylene oxide and isobutylene oxide units, or mixtures thereof. Nonlimiting examples of suitable starter alcohols are long-chain alkanols or phenols substituted by long-chain alkyl in which the long-chain alkyl radical is in particular a straight-chain or branched C₆-C₁₈-alkyl radical. Preferred examples include tridecanol and nonylphenol.

Further suitable synthetic carrier oils are alkoxylated alkylphenols, as described in DE-A-10 102 913.

Further customary additives are corrosion inhibitors, for example based on ammonium salts of organic carboxylic acids, said salts having a tendency to form films, or on heterocyclic aromatics in the case of nonferrous metal corrosion protection; antioxidants or stabilizers, for example based on amines such as p-phenylenediamine, dicyclohexylamine or derivatives thereof, or on phenols such as 2,4-di-tert-butylphenol or 3,5-di-tert-butyl-4-hydroxyphenylpropionic acid; demulsifiers; antistatics; metallocenes such as ferrocene; methylcyclopentadienylmanganese tricarbonyl; lubricity improvers (lubricity additives) such as particular fatty acids, alkenylsuccinic esters, bis(hydroxyalkyl) fatty amines, hydroxyacetamides or caster oil; and dyes (markers). If appropriate, it is also possible to add amines to lower the pH of the fuel.

The components or additives can be added to the fuel compositions individually or as a previously prepared concentrate (additive package) together with the inventive polyisobuteneamines I.

The inventive polyisobuteneamines of the general formula I are added to the fuel compositions typically in an amount of from 5 to 5000 ppm by weight, preferably from 10 to 2000 ppm by weight, especially from 25 to 1000 ppm by weight, in particular from 50 to 500 ppm by weight, in each case specified as the pure substance content (i.e. without solvent and diluent) and based on the total amount of the fuel composition. When further detergent additives with polar moieties (a) to (i) are also used, the dosages specified above are based on the total amount of all fuel detergents including the inventive polyisobuteneamines I. The other components and additives mentioned are, if desired, added in amounts customary therefor.

The present invention will now be illustrated in detail with reference to the nonlimiting working examples which follow:

### PREPARATIVE EXAMPLES

### Example 1

### Preparation of a Polyisobuteneamine “P1” from a Polyisobutene Having a Number-Average Molecular Weight (Mₙ) of 720

In analogy to preparative example 2 from {2}, 500 g of a high-reactivity polyisobutene, prepared from pure isobutene, with a number-average molecular weight (Mₙ) of 720 and a proportion of terminal vinylidene double bonds of 81 mol %, 180 g of a solvent mixture composed of n-paraffins/naphthenes and 2.8 g of cobalt octacarbonyl were heated at 185° C. in a 2.5 l lifting stirrer autoclave with stirring at 280 bar of CO/H₂ (1:1 vol./vol.) for 5 hours. Subsequently, the mixture was cooled to room temperature, the catalyst was removed with 400 ml of 10% by weight aqueous acetic acid and the mixture was washed to neutrality. The resulting oxo product is treated with 1 l of ammonia, 300 g of ethanol and 100 g of Raney cobalt in a 5 l roller autoclave under a hydrogen pressure of 200 bar at 180° C. for 5 hours. After the mixture had been cooled, the catalyst was filtered off, excess ammonia was evaporated off and the solvent was distilled off. This resulted in 520 g of a corresponding polyisobuteneamine with a terminal —CH₂NH₂— moiety with a kinematic viscosity of 98 cSt, measured in undiluted form at 100° C. in an Ubbelohde viscometer.

### Application Examples

In the application examples which follow, for comparison, a polyisobuteneamine “P2” formed from a homologous high-reactivity polyisobutene, prepared from pure isobutene, with a number-average molecular weight (Mₙ) of 1000 with a terminal —CH₂NH₂— moiety was used in each case. P2 had a kinematic viscosity of 241 cSt, measured in undiluted form at 100° C., in an Ubbelohde viscometer.

### Examples 2a-2e

### Intake Valve Cleanliness in Gasoline Engines

The testing of the intake valve cleanliness in gasoline engines was carried out with a Mercedes Benz M 111 test engine to CEC F-20-A-98 (in example 2a) or a Mercedes Benz M 102E test engine to CEC F-05-A-93 (in examples 2b-2e). The base fuel used was a Eurosuper fuel to EN 228. The deposits were measured on the four intake valves, from which the mean was formed in each case. In examples 2a and 2b, in each case only the pure polyisobuteneamines P1 and P2 were metered in, and in examples 2c-2e in each case commercial additive packages or reproductions of commercial additive packages which additionally comprised—as well as further coadditives in a small amount, but which exert no influence on the intake valve cleanliness—polyether carrier oils. The doses of the particular additives specified in ppm by weight (reported as pure substance content, without solvent) are based in each case on the total amount of the gasoline fuel formulation used. Table 1 which follows shows the results of the measurements.

TABLE 1
Measurements of intake valve cleanliness

| | | Mean of the depositions |
|---|---|---|
| Examples | | in mg/valve |
| 2a | Base value (fuel without additives) | 154 |
| | P1 (137 ppm by weight) | 33 |
| | P2 (137 ppm by weight) | 16 |
| 2b | Base value (fuel without additives) | 313 |
| | P1 (109 ppm by weight) | 39 |
| | P2 (109 ppm by weight) | 54 |
| 2c | Base value (fuel without additives) | 518 |
| | P1 (130 ppm by weight) + | 13 |
| | T1 (155 ppm by weight) | |
| | P2 (130 ppm by weight) + | 20 |
| | T1 (155 ppm by weight) | |
| 2d | Base value (fuel without additives) | 313 |
| | P1 (118 ppm by weight) + | 8 |
| | T1 (49 ppm by weight) | |
| | P2 (118 ppm by weight) + | 13 |
| | T1 (49 ppm by weight) | |
| 2e | Base value (fuel without additives) | 313 |
| | P1 (70 ppm by weight) + | 78 |
| | T1 (54 ppm by weight) | |
| | P2 (70 ppm by weight) + | 56 |
| | T1 (54 ppm by weight) | |

“T1” is a commercial polyether carrier oil with the structure of a tridecanol reacted with 22 mol of butylene oxide.

It is clearly evident from examples 2a-2e that, within the range of customary scatter of the results, on the basis of the measurement inaccuracy of the method, a comparable efficacy in keeping the intake system clean is present when the inventive polyisobuteneamine P1 is used to that in the case of the prior art polyisobuteneamine P2.

### Examples 3a and 3b

### Valve Sticking Performance

The testing of the valve sticking performance was undertaken by tests in the VW Wasserboxer test to CEC F-16-T-96. The base fuel used was a Eurosuper fuel to EN 228. The criteria of the test method were used to test for a “pass” (no valve sticking in three successive test runs) or a “fail” (valve sticking in the first, second or third of the successive test runs). Valve sticking becomes noticeable here by virtue of the engine starting only with a delay, if at all. In order to enable a differentiation, testing was deliberately effected in the boundary range of expected valve sticking. The doses of the particular additives specified in ppm by weight (reported as pure substance content, without solvent) are based in each case on the total amount of gasoline fuel formulation used. The two tables which follow show the results of the tests.

TABLE 2
Example 3a - Valve sticking tests with pure polyisobuteneamines

| | |
|---|---|
| P2 (80 ppm by weight), for comparison | Fail (sticking in the 2nd test run) |
| P1 (80 ppm by weight), inventive | Pass |
| P1 (160 ppm by weight), inventive | Fail (sticking in the 1st test run) |

Compared to P2, the inventive P1 is less prone to valve sticking at the same dosage. The fact that valve sticking fundamentally cannot be eliminated is shown by the test with 160 ppm by weight of P1. For this reason, carrier oil is always also used in practice.

TABLE 3
Example 3b - Valve sticking tests with
polyisobuteneamine-carrier oil mixtures

| | |
|---|---|
| P1 (154 ppm by weight) + | Pass |
| T1 (15 ppm by weight) | |
| P2 (154 ppm by weight) + | Fail (Sticking in the 1st test run) |
| T1 (15 ppm by weight) | |
| P2 (154 ppm by weight) + | Fail (Sticking in the 1st test run) |
| T1 (30 ppm by weight) | |
| P2 (154 ppm by weight) + | Pass |
| T1 (45 ppm by weight) | |

“T1” is a commercial polyether carrier oil with the structure of a tridecanol reacted with 22 mol of butylene oxide.

Valve sticking can be prevented by adding carrier oil, but three times the amount of carrier oil are required for this purpose for P2 of the prior art compared to that required for the inventive P1.

### Example 4

### Mixing Tests of Compatibility of Detergents with Carrier Oils at Low Temperatures

The compatibility and storage stability of polyisobuteneamines and polyether carrier oils were tested at 20° C. (room temperature), 0° C. and −20° C. To this end, in each case 60 parts by weight of a 50% by weight solution of P1 or P2 in a hydrocarbon mixture customary for this purpose, as the diluent, were mixed with 40 parts by weight of the polyether carrier oil T2 or T3 at the temperatures specified, and the homogeneity of the mixture was assessed visually. “T2” is a commercial polyether carrier oil with the structure of a tridecanol reacted with 15 mol of propylene oxide; “T3” is a commercial polyether carrier oil with the structure of a tridecanol reacted with 30 mol of propylene oxide. The propylene oxide-based carrier oils used are known for the fact that a slight degree of phase separation occurs at low temperatures, and a slight degree of cloudiness occurs even at room temperature. These undesired effects have to be remedied in practice by addition of in some cases considerable amounts of additional solvent, for example xylene. The results of the mixing tests are compiled in the table which follows.

TABLE 4
Mixing tests of polyisobuteneamines with polyether carrier oils

| | 20° C. | 0° C. | −20° C. |
|---|---|---|---|
| P1 + T2 | clear solution | clear solution | clear solution |
| P2 + T2 | clear solution | clear solution | phase separation |
| P1 + T3 | clear solution | clear solution | clear solution |
| P2 + T3 | cloudy | phase separation | phase separation |

The results show the significantly better compatibility of the inventive P1 with the polyether carrier oils compared to P2 of the prior art.

### Example 5

### Mixing Tests of Improvement in the Compatibility of Polyisobuteneamine in a Mixture of Mineral Gasoline Fuel with Ethanol

The influence of polyisobuteneamines on the improvement of compatibility in a mixture of mineral gasoline fuel with ethanol with regard to the production of “E85” fuel was tested using P1 and P2. To this end, equivalent amounts of in each case 0.1 g of P1 or P2 (pure substance, without solvent) were predissolved in 30 ml of unadditivized Eurosuper fuel to EN 228 (“GF”) (such high “dosages” are unusual in practice; in other words, the cloudiness which occurs here will be significantly lower with dosages customary in practice). Thereafter, the mixture was made up to 200 ml with ethanol, which corresponds approximately to the composition of the “E85” fuel. The sample was observed for occurrence of noticeable cloudiness. The table which follows shows the results of the test.

TABLE 5
Mixing tests of gasoline fuel with ethanol

| | Cloudiness on | Volume ratio of | |
|---|---|---|---|
| | addition of | ethanol to GF | Final state in “E85” |
| P1 | 120 ml of ethanol | 4:1 | slight cloudiness |
| P2 | 60 ml of ethanol | 2:1 | high cloudiness |

These results show the clearly strong influence of the inventive P1 on the improvement of the compatibility of polyisobuteneamine in a mixture of mineral gasoline fuel with ethanol compared to P2 of the prior art. While noticeable cloudiness occurs with P2 even at a volume ratio of ethanol:GF of 2:1, the volume ratio of ethanol:GF can be increased for P1 up to 4:1 before cloudiness occurs. In the “E85” fuel too (volume ratio of ethanol:GF=5.7:1), P1 exhibits significantly lower cloudiness.

### Example 6

### Influence of the Viscosity of the Polyisobuteneamine on the Flow Performance

The advantage of a lower-viscosity polyisobuteneamine with regard to better flow performance through apparatus and lines becomes evident by the amount of solvent or diluent which is required for the throughput of the same absolute amount of polyisobuteneamine within the same time unit. In a typical production process for P2 (kinematic viscosity of 241 cSt, undiluted at 100° C.), adjustment of the dilution of the end product with a customary hydrocarbon mixture to a polymer content of 65% by weight resulted in the same volume flow per unit time as in the analogous production process for the inventive P1 (kinematic viscosity of 98 cSt, undiluted at 100° C.) at an adjustment of the dilution of the end product with the same hydrocarbon mixture to a polymer content of 71% by weight. This means a production rise for P1 of 9% of active polymer, dissolved in less diluent.

1. A polyisobuteneamine represented by formula I
in which
R¹—CH₂—NR²R³ (I)
the variable R¹ is a polyisobutyl radical which is derived from isobutene and up to 20% by weight of n-butene and has a number-average molecular weight Mₙ of from 600 to 770, and
the variables R² and R³ are each independently hydrogen, a C₁-C₁₈-alkyl, C₂-C₁₈-alkenyl, C₄-C₁₈-cycloalkyl, C₁-C₁₈-alkylaryl, hydroxy-C₁-C₁₈-alkyl, poly(oxyalkyl), polyalkylenepolyamine or polyalkyleneimine radical or, together with the nitrogen atom to which they are bonded, are a heterocyclic ring, which is obtained by a process comprising reacting a polyisobutene which has at least one of the following properties:
[a]proportion of vinylidene double bonds of at least 60 mol %, based on the polyisobutene;
[b] content of isobutene units in the polyisobutene polymer skeleton of at least 85% by weight;
[c] polydispersity of from 1.05 to 7
with carbon monoxide and hydrogen in a hydroformylation reaction in the presence of a hydroformylation catalyst, and subjecting the oxo intermediate thus prepared to a reductive amination in the presence of hydrogen, of a suitable nitrogen compound and of a suitable catalyst.

2. The polyisobuteneamine of claim 1, in which the variable R¹ has a number-average molecular weight Mₙ, of from 700 to 730.

3. The polyisobuteneamine of claim 1, in which the —NR²R³ moiety has been obtained from ammonia or a polyamine of the general formula II
H₂N—(CH₂CH₂—NH—)ₙ—H (II)
in which the variable n is an integer from 1 to 5.

4. The polyisobuteneamine of claim 1 with a kinematic viscosity of from 70 to 200 cSt, measured in undiluted form at 100° C.

5. A fuel composition comprising at least one polyisobuteneamine according to claim 1 in an amount of from 5 to 5000 ppm by weight.

6. The fuel composition according to claim 5, comprising from 0.1 to 95% by volume of C₁-C₄-alkanols, based on the total weight of the composition.

7-8. (canceled)

9. A process of preparing the polyisobuteneamine of claim 1, comprising:
reacting a polyisobutene which has at least one of the following properties:
[a]proportion of vinylidene double bonds of at least 60 mol %, based on the polyisobutene;
[b] content of isobutene units in the polyisobutene polymer skeleton of at least 85% by weight;
[c] polydispersity of from 1.05 to 7
with carbon monoxide and hydrogen in a hydroformylation reaction in the presence of a hydroformylation catalyst, and subjecting the oxo intermediate thus prepared to a reductive amination in the presence of hydrogen, of a suitable nitrogen compound and of a suitable catalyst
